(19) United States
(12) Patent Application Publication
(10) Pub. No.: US 2007/0074990 A1
(43) Pub. Date: Apr. 5, 2007

(54) Lipid retaining container

(75) Inventors: Barbara L. Merboth, Bridgewater, NJ (US); Florence Stoffel, Flemington, NJ (US); Malcolm Will, Aberdeen, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/239,343

(22) Filed: Sep. 30, 2005

(51) Int. Cl. A61B 19/02

(52) U.S. Cl. 206/438

(57) ABSTRACT

A sterile container assembly for storing sterile allograft tissue implant material is constructed to hold allograft bone tissue material mounted in a sterile blister package allowing drainage of the lipids contained in the implant without destroying the seal and sterility of the package. The package for storing sterile allograft tissue implant forms is constructed with an outer blister tray defining an open faced cavity and a flange extending outward from the tray cavity surrounding the cavity and an inner blister tray seated in the outer blister tray cavity. The inner blister tray also defines an open faced cavity and a flange extending outward from the cavity and a two piece implant insert having a recess allowing lyophilization while preventing lipid flow is mounted in the inner blister tray cavity. Lids are provided for both the inner and outer blister trays.

### CROSS-REFERENCE TO RELATED APPLICATIONS

There are no other applications related to the present application.

### TECHNICAL FIELD OF THE INVENTION

The present invention generally relates to packaging for sterile tissue specimens for use in tissue transplant and more specifically to packages for allograft implant tissue forms in a specifically designed sterile package for allograft bone implants that have a high lipid content which allows moisture to escape from the tissue during lyophilization while preventing lipids from flowing out.

### BACKGROUND OF THE PRIOR ART

Allograft tissue forms are useful in orthopaedic surgery. In practice, processed human tissue is delivered to the hospital and eventually to the surgeon in a form useful for surgical implantation in a sterile package. Unfortunately, while large amounts of time have been spent on development and processing of tissue implant forms and materials, little consideration has been spent on the package design and the specific problems involved with packaging bone tissue material. Packaging that is currently used for bone implant forms is cumbersome and it is often difficult to easily remove the implant form from such packaging while wearing gloves during a surgical procedure.

Furthermore, a need has developed for a simple, inexpensive packaging that may be used to safely retain implant allograft bone tissue material in a sterile condition away from the lipid pooling while allowing storage of same and maintaining sterility up until the actual time of implantation during surgery. While tissue processing removes most of the lipids found in bone, some other tissue such as cortical fibular shafts have a high lipid content and not all of the lipids are removed during the tissue processing. Over time these lipids drain from the tissue and are deposited onto the package and can be absorbed by or cause discoloration of the packaging material. If the package material forms part of the package seal and that material absorbs the lipids, the seal will be destroyed causing failure of the sterile barrier. When the material is only discolored or does not form part of the sterility seal, the result is a visually unattractive package, which while causing no harm to the package or tissue gives the appearance that the product is damaged or spoiled.

There are presently available a number of kinds of packaging for sterile specimens. One form of commonly used packaging is to provide the allograft tissue in a freeze-dried state in a glass jar or bottle with a specifically designed stopper. The aseptically processed tissue is placed into the glass jar, and the stopper is placed on top of the jar. The stopper is designed so that it sits on top of the jar and there is a gap that allows the moisture to escape during the lyophilization process. The jars are then placed into a commercial freeze dryer with the freeze drying process occurring under vacuum. At the end of the cycle while the jars are still under vacuum, a mechanism in the freeze dryer presses the stopper into the jar and creates a seal between the jar and the stopper that maintains the vacuum in the jar. The jars are then removed from the freeze dryer and the closed stopper is secured in place with a metal crimp and plastic lid. This package is effective in allowing the moisture to be removed from the tissue, and in protecting the tissue. However there are several drawbacks to this design. The first drawback is since the stopper is in the up position for lyophilization and since many donors are processed in one freeze drying cycle, there is a potential for cross contamination between containers. Secondly, the jars are subject to breakage during shipping and thirdly, the metal crimp cap is difficult to remove and can tear the gloves of the operating room personnel opening the container, causing possible contamination and the need to destroy the tissue.

Another form of packaging which has been used for holding products such as a pre-sterilized medical devices and allograft implant forms are sealed blister containers. Such containers generally comprise a relatively rigid blister tray with a peripheral flange and a foil composite material or paper backing sheet positioned over the open tray in overlapping engagement with the peripheral flange forming a cover. The cover is manually peeled away from the flange to which it is sealed to allow access to the medical device or implant tissue form contained therein. Other examples of packaging for medical devices or implant forms which have been utilized or are known in the art are shown in the following patents.

U.S. Pat. No. 6,012,580 is directed toward a clamshell type package with two halves pivoted together at a hinge constructed of a transparent plastic constituted to serve as a universal package for implant materials.

U.S. Pat. No. 5,720,391 shows a blister package and insert holder for a heart valve prosthesis. The package is constructed with an outer tray which receives an inner tray. An inner tray lid seals the inner tray and is provided with a pull tab on its exterior surface so that the same can be pulled away from the lip of the inner tray for access to the sterile heart valve prosthesis. The outer tray is provided with a tray lid which is sealed to the lip of an outer flange of the outer tray.

U.S. Pat. No. 5,690,226 shows a sealed air tight molded blister package of PETG having a hollow interior with the opening surrounded by an outwardly extending flange. A multi-layer peelable cover is sealed to the flange surrounding the opening. The cover and the flange are deformed towards the bottom of the container from the plane of the opening sealed by the peelable cover.

U.S. Pat. No. 5,615,770 discloses a sterilizable medical implant package insert placed within a standard sterile implant package holding an implant and allowing for the automatic presentation of the implant from the insert when the sterile package top is opened.

U.S. Pat. No. 5,257,692 is directed toward a three envelope package for preserving tissue specimens or other sterile objects. The sterile tissue sample is sealed within an innermost envelope which is sterile inside and outside. The innermost envelope is sealed within the sterile interior of an intermediate envelope which is both sterile inside and outside. This intermediate envelope is made of foil or another substance impermeable to a storage medium such as liquid nitrogen and is sealed within the sterile interior of an outermost envelope made of foil or another substance impermeable to a storage medium. The outermost envelope provides complete impermeability to liquid nitrogen, eliminating the possibility of nitrogen seepage through the peel-baked seal of the intermediate envelope.

U.S. Pat. No. 5,176,258 shows a package with a peripheral flange around a blister defining an open cavity for receiving a product and a compressible insert for securing the product against movement in the package cavity. At least one projection on the insert extends laterally from the cavity over the peripheral flange and a lid covering the cavity is continuously sealed to the peripheral flange and the projection along a single seal.

U.S. Pat. No. 4,750,619 is directed toward a package for a sterile prosthetic implant element comprising an outer receptacle and an inner receptacle which fits into the cavity of the outer receptacle. Both of the receptacles are provided with outwardly extending flanges surrounding the outer periphery of the cavity of each receptacle and receive a lid which is secured thereto. A tray received within the inner receptacle has hinged leafs so that upon placement of the tray within the receptacle and folding the hinged leafs, the prosthetic implant element is confined within a defined envelope to protect the same against damage.

U.S. Pat. No. 4,697,703 discloses a double sterile package for medical items such as a hip joint prosthesis with an outer open container enclosing an inner package containing the medical item. The inner package has a first insert contained in the open top container, a lid and a second insert carried by the lid. The outer container is sealed with a cover which engages an outer peripheral flange formed around the open top of the outer container.

U.S. Pat. No. 6,830,149 by the present inventor, discloses a package for storing sterile allograft tissue implant forms constructed with an outer container defining an open faced cavity and a flange extending outward from said cavity with a stepped recess formed in said flange surrounding the cavity. An inner container which is adapted to be seated in the cavity of the outer container defines an open faced cavity and a flange extending outward from said cavity, the inner container flange being of a dimension to fit into the stepped recess of the outer container. An insert member sized to fit into said inner container cavity defines a shaped depression therein to hold a tissue implant form within a designated space defined by the shaped depression. The inner container is covered by a permeable cover sealed to the flange of the inner container allowing the tissue form to be treated and the outer container is covered by an impermeable cover sealed to the flange of the outer container covering the outer container cavity. This design while effective for many tissue forms in keeping lipids away from the lid is not adequate for tissue forms that have high lipid content such as fibular shafts. The lipids in such tissue could then damage the seal of the package and compromise the sterility of the package.

As noted, none of the above identified packages are conducive to bone tissue forms having a high lipid content such as fibular shafts. Human bone tissue forms, after processing, retain lipids sometimes called structural fats. Lipids are a group of fatty substances that include fatty oils, waxes, sterols such as cholesterol, triglycerides (the principal forms of fat in body fat) and esters of fatty acids containing groups such as phosphoric acid (phospholipids) and carbohydrates (glycolipids). Over time, the fatty oils contained in the processed bone material are drawn out of the bone and are absorbed by the permeable cover and begin to dissolve the adhesive bond between the cover and the container. This activity causes the cover to separate from the container receptacle destroying the sterility of the package and leaving pools or rivets of a dark oily material which is unsightly and causes the surgeon to discard or send back the tissue material. This lipid migration is a serious problem for manufacturers of allograft bone material, and presents problems for the hospital and surgeon user. Accordingly, the present container assembly has been developed for allograft bone material and storage of the same to contain the lipids and prevent the same from compromising the package seal thus further preventing cross-contamination or potential loss of sterility.

### SUMMARY OF THE INVENTION

The present invention is directed toward an allograft tissue insert container constructed to hold allograft bone tissue material having high lipid content is mounted in a sterile blister package allowing drainage of the lipids contained in the implant without destroying the seal and sterility of the package while still allowing lyophilization. The package for storing sterile allograft tissue implant forms is constructed with an outer blister tray defining an open faced cavity and a flange extending outward from said cavity and surrounding the cavity, an inner blister tray seated in the outer blister tray cavity. The inner blister tray also defines an open faced cavity and a flange extending outward from the cavity. A two piece implant insert is mounted in the inner blister tray cavity. Lids are provided for both the inner and outer blister trays.

It is an object of the invention to provide for an implant insert that will contain excess lipids released from the bone tissue allowing moisture to exit through vents during freeze drying process.

It is another object of the invention to provide a packaging system which holds sterile bone tissue material in a sterile condition which allows a user easy access to the sterile bone tissue material.

It is yet another object to provide a sterile package for bone tissue material which is resistant to cracking or shattering or loss of strength during transportation and during storage.

It is another object of the invention to provide a medical package in which an implant tissue form can be maintained in a sterile condition when it is presented to the surgeon for implantation.

It is still another object of the invention to provide a package for storing sterile bone tissue material for later use in an implant situation.

It is yet another object of the invention to provide a package for storing bone tissue implant material whereby the bone tissue implants material can be removed from the package in a sterile condition.

In the accompanying drawings, there is shown illustrative embodiments of the invention from which these and other objectives, novel features and advantages will be readily apparent.

### BRIEF DESCRIPTION OF THE DRAWINGS

FIG. 1 is a perspective view of the lipid retaining container;

FIG. 2 is an exploded perspective view of the container of FIG. 1 showing the cover, the pull tab cover, the tissue insert container, the tissue insert container blister pack and the outer blister pack in spaced relationship;

FIG. 3 is a side elevation view of the outer blister pack of the lipid retaining container;

FIG. 4 is top plan view of the outer blister pack of the tissue package of FIG. 3;

FIG. 5 is a side elevation view of the inner blister pack of the lipid retaining container;

FIG. 6 is a top plan view of the inner blister pack of FIG. 5 with the cover removed;

FIG. 7 is a top plan view of the pull tab cover of the inner blister pack shown in FIGS. 5 and 6;

FIG. 8 is an exploded spaced view of the implant insert container;

FIG. 9 is a top plan view of the bottom member of the implant container shown in FIG. 8;

FIG. 10 is a bottom plan view of the bottom member of the implant container shown in FIG. 9;

FIG. 11 is a top plan view of the top member of the implant insert container;

FIG. 12 is a bottom plan view of the of the bottom member of the implant insert container; and

FIG. 13 is a side elevational view of the assembled insert container holding an allograft bone implant structure.

### DETAILED DESCRIPTION OF THE INVENTION

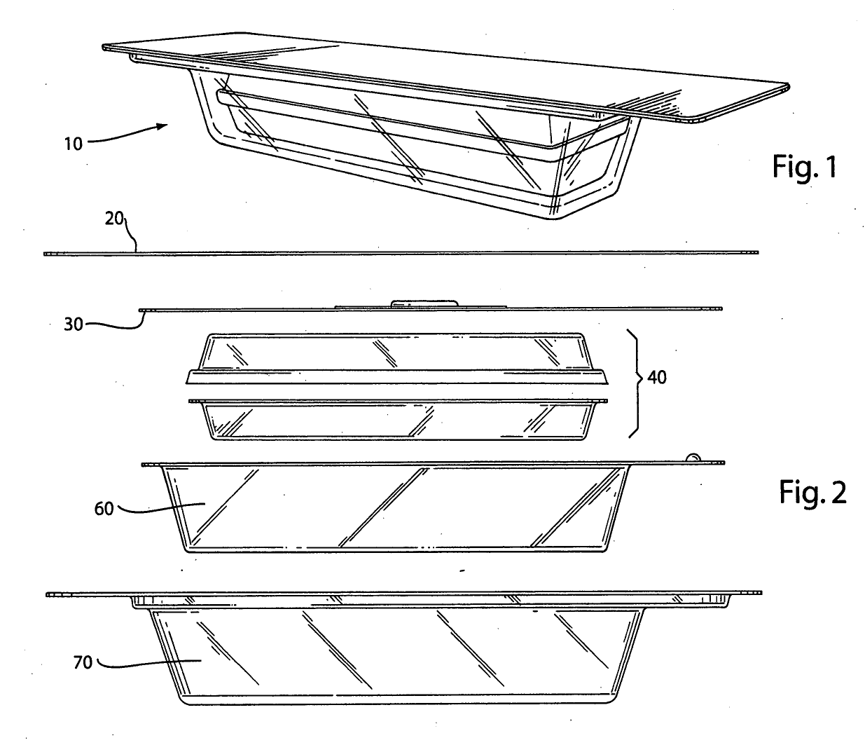

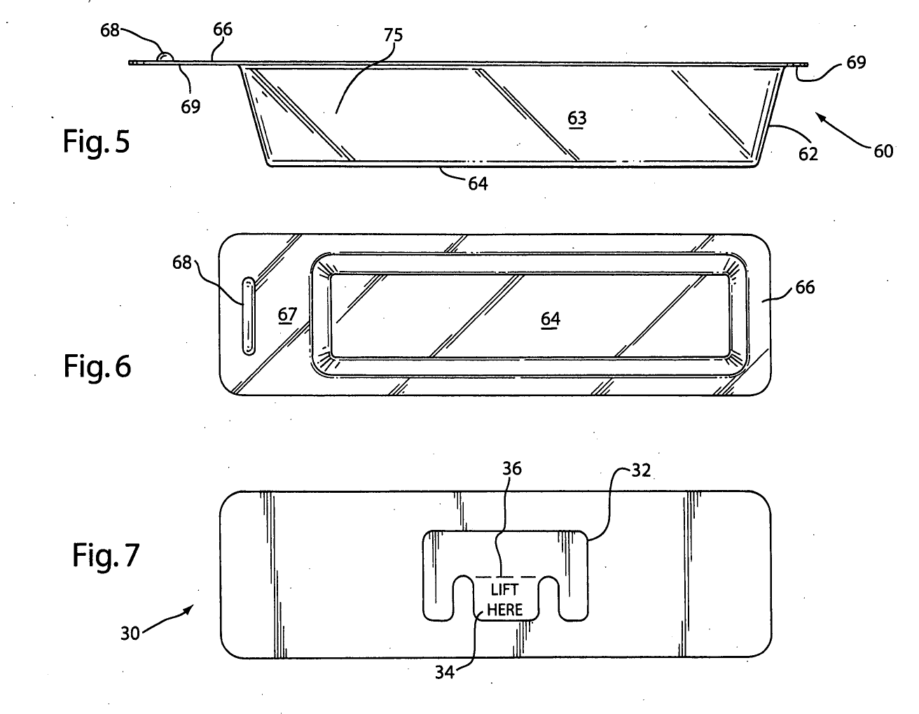

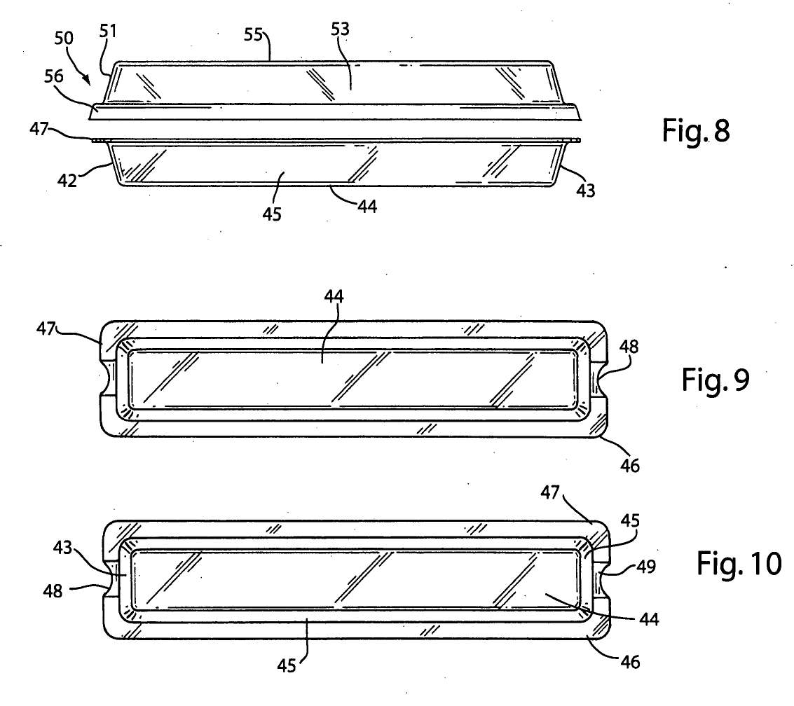

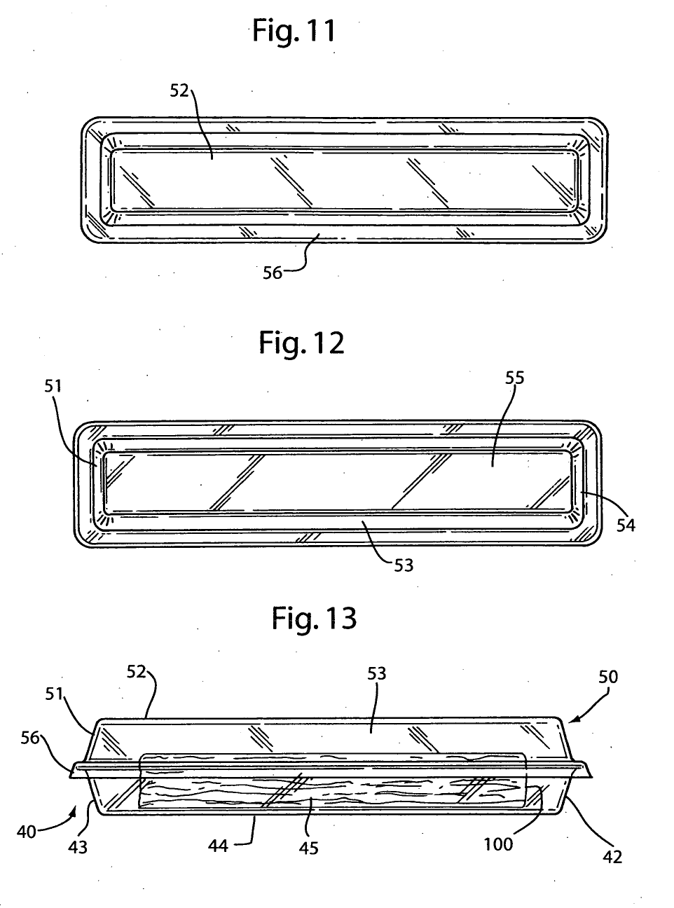

The preferred embodiment and best mode of the tissue form package invention is shown in FIGS. 1-13. The tissue package 10 shown in FIG. 1 and exploded view in FIG. 2 comprises an outer blister pack 70 with a cover 20, an inner blister pack 60 with a pull off cover 30 and a two piece implant insert container 40.

The implant insert container 40 as best shown in FIGS. 8 through 12, is constructed with a PTEG lower or tissue seating member 42 which fits into a step 54 formed in the flange 56 upper member 50. The two sections are held together by the construction of the inner tray and its associated lid assembly. The upper member 50 is constructed with outwardly angled end walls 51 and integral outwardly angled side walls 53 extending downward from a planar top 55. An angular “L” shaped flange 56 extends angularly outward from the end walls 51 and side walls 53 and forms a planar seat 54 which is substantially parallel to the top 55 upon which flange 46 of the lower member 42 is seated. The lower member 42 has outwardly angled walls 43, side walls 45 and a planar base 44. The end walls and side walls upper portion are extended outwardly from the plane of the end walls and side walls and are positioned parallel to the plane of the base 44 to form a flange 46. Both ends of flange 46 are notched at 48 and slightly elevated above the plane of the flange 46 into the respective sidewall to form a moisture vent 49. The design of the angled flange 46 gives the container lipid retention while allowing moisture release. The implant insert container 40 which holds allograft bone structure 100 is placed in an inner blister tray 60 shown in FIGS. 5-7.

The inner blister tray 60 has outwardly angled end walls 62 and outward angled side walls 63 from the integral planar base 64. A flange 66 extends outward from upper portion of the end walls and side walls and surrounds the cavity 65 with one end 67 of flange 66 extending farther out from the container to provide a grasping handle. The end 67 is also provided with a rib 68 allowing for easy removal of a TYVEK® cover 30 which is sealed to the top planar surface of flange 66. The cover 30 is provided with a base area 32 having a pull tab 34 mounted to the base area 32 which can be raised along perforated line 36.

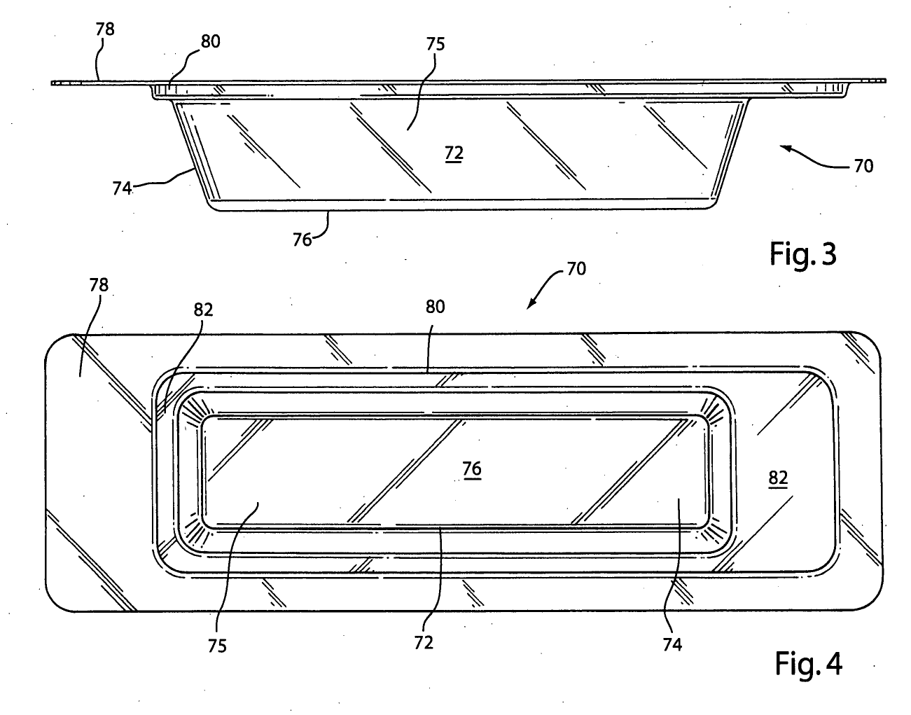

The inner tray 60 which holds the insert container 40 is seated in an outer blister pack 70 shown in FIGS. 3 and 4. The outer blister tray 70 has inwardly angled side walls 72 and inwardly angled end walls 74 as viewed from the top to the base and are integrally formed with a planar base 76 to define a cavity 75. A flange 78 extends outwardly from the end walls and side walls and has a recess 80 formed therein, which defines a step 82. The recess 80 holds and supports the bottom surface 69 of the flange 66 of the inside tray 60.

The component material used for the blister trays and implant container of the allograft bone tissue package assembly 10 are preferably made of an available material such as polytheylene terephthalateglycol (PETG) (a copolyester made by Eastman Chemical). The outer blister tray 70 is a laminate with the inner layer being PETG and the outside layer being another available material polycholrotrifluoroethylene (PCTFE) sold under the trademark ACLAR (a fluorinated-chlorinated thermoplastic made by Allied Corporation) which is impermeable to oxygen and other atmospheric gases and which is a highly moisture resistant barrier.

The lids or covers 20 and 30 are sized and configured to cover cavities of outer blister tray 70 and the inner blister tray 60 respectively. The cover for the inner tray 60 is preferably constructed of TYVEK® and the outer laminated tray 70 has a foil cover. Each cover is seated on the upper planar surface of respective flange of each container covering the container cavity and extends to the peripheral edge of each container.

The tissue 100 is placed in the lower half of the insert container member 42 and then covered by the upper member 50 with the flange 56 extending down past the periphery of the edge of the flange 46 of the lower member. The entire unit is placed in the inner blister tray 60 which is then sealed with a TYVEK® cover 30 and the container is lyophilized. After lyophilization the inner tray 60 is in turn placed in an outer blister tray 70 which is then sealed with a foil cover or lid 20 forming an outer moisture barrier assembly. During the lyophilization process of the inner tray 60 the TYVEK® cover is facing up and the excess lipids will collect in the bottom of the container. Since during shipping and storage the unit could be oriented in any direction allowing the lipids to flow about the inside of the container, the top and bottom portion of the container interlock. The container is designed in such a manner that the lipids can flow within the container but not travel outside the container and onto the TYVEK® cover.

The packaging is easily disassembled during surgery by simply removing the cover of the outer container, removing the inner container and pulling the cover away from it and removing the insert container allowing the implant construct to be removed from it for implantation into the patient. In addition using a clear packaging provides product visibility allowing easy identification of the product. Furthermore the packaging of the present invention provides dual sterile barriers in a recyclable container.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present inventions defined by the following claims.

What I claim is:

1. A container assembly for storing sterile allograft tissue implant material having lipids retained therein in a sterile condition comprising:
an outer tray constructed with end walls, side walls and a base wall defining an open faced cavity and a flange extending outward from said cavity and a cover for said outer tray,
an inner tray mounted in said outer tray cavity, said inner tray being constructed with end walls, side walls and a base wall defining an open faced cavity and a flange extending outward from said cavity and a cover for said inner tray,
an implant insert container sized to fit into said inner tray cavity, said implant insert container comprising a bottom member with side walls and end walls integrally connected to a base wall to define a cavity and a flange extending outward from said cavity, a top member with side walls and end walls integrally connected to a top wall to define a cavity, a flange located on the end of said side walls and end walls extending away from said cavity and being dimensioned to fit over said bottom member flange.

2. A container assembly as claimed in claim 1 wherein outer tray is a blister tray and has a foil cover over said outer tray cavity sealing said cavity from the outside atmosphere.

3. A container assembly as claimed in claim 1 wherein inner tray is a blister tray and has a TYVEK® cover over said inner tray cavity maintaining sterility.

4. A container assembly as claimed in claim 3 wherein said cover has a base area and a pull tab mounted to said base area.

5. A container assembly as claimed in claim 1 wherein said end walls and side walls of said bottom member and said top member are angled.

6. A container assembly as claimed in claim 1 wherein said outer tray flange defines a recess therein to seat the flange of said inner tray.

7. A container assembly as claimed in claim 1 wherein said inner tray and insert container are constructed of PETG.

8. A container assembly as claimed in claim 1 wherein said outer tray cover is a laminate of PETG and PCTFE.

9. A container assembly as claimed in claim 1 wherein said flange of said outer tray has one end which extends outward further than the other portions of said flange to form a handle for the respective container.

10. A container assembly as claimed in claim 1 wherein said flange of said inner tray has one end which extends outward further than the other portions of said flange to form a handle for the respective container.

11. A container assembly as claimed in claim 10 wherein said one end of said inner tray handle has a rib formed thereon.

12. A container assembly as claimed in claim 1 wherein said implant insert container comprises a two piece implant insert sized when the two pieces are mated together to fit into said inner container cavity, said implant insert comprising a bottom member with side walls and end walls integrally connected to a base wall to define a cavity and a flange extending outward from and around the cavity, said end walls defining a notched recess allowing communication of moisture outside of the insert cavity, a top member with side walls and end walls integrally connected to a top wall to define a cavity, a flange extending outward from said side walls and end walls, said flange defining a step therein which fits over said flange of said bottom member.

13. A container assembly for storing sterile allograft tissue implant material retained therein in a sterile condition comprising:
an outer tray constructed with angled end walls, angled side walls and a planar base defining an open faced cavity and a flange extending outward from said cavity, said flange defining a recess therein which surrounds said cavity, and a lid for said outer tray;
an inner tray mounted in said outer tray cavity constructed with end walls, side walls and a planar base defining an open faced cavity and a flange extending outward from said cavity, said flange being dimensioned to fit in the recess of said outer tray flange;
a two piece implant insert sized when the pieces are mated together to fit into said inner container cavity, said implant insert container comprising a bottom member with side walls and end walls integrally connected to a base wall to define a cavity and a flange extending outward from and around the cavity, said end walls defining a recess allowing communication of fluids outside of the insert container cavity, a top member with side walls and end walls integrally connected to a top wall to define a cavity, a flange extending outward from said side walls and end walls, said flange defining a step therein which fits over said flange of said bottom member.

14. A container assembly as claimed in claim 13 wherein said inner tray and insert container are constructed of PETG.

15. A container assembly as claimed in claim 13 wherein said outer tray is a laminate of PCTFE and PETG.

16. A container assembly as claimed in claim 13 wherein outer tray is a blister tray and said lid is constructed of foil, said lid being placed over said outer cavity sealing said cavity from the outside atmosphere.

17. A container assembly as claimed in claim 13 wherein inner tray is a blister tray and has a TYVEK® lid over said inner cavity sealing said cavity and forming a sterile barrier.

18. A container assembly as claimed in claim 13 wherein inner tray has a lid over said inner cavity sealing said cavity from the outside atmosphere, said lid being provided with a reinforced area and a pull tab mounted to said reinforced area.

19. A container assembly for storing sterile allograft tissue implant material retained therein in a sterile condition comprising:
an outer tray constructed with angled end walls, angled side walls and a planar base defining an open faced cavity and a flange extending outward from said cavity, said flange defining a recess therein which surrounds said cavity, said flange of said outer tray having one end which extends outward further than the other portions of said flange to form a handle for the respective tray; and a lid for said outer tray covering said cavity;
an inner tray mounted in said outer tray cavity constructed with end walls, side walls and a planar base defining an open faced cavity and a flange extending outward from said cavity, said flange being dimensioned to fit in the recess of said outer tray flange, said flange of said inner tray having one end which extends outward further than the other portions of said flange to form a handle for the respective container, a lid for said inner tray covering said cavity;
a two piece implant insert container sized when the pieces are mated together to fit into said inner container cavity, said implant insert container comprising a bottom member with side walls and end walls integrally connected to a base wall to define a cavity and a flange extending outward from and around the cavity, said end walls defining a notched recess allowing communication of fluids outside of the insert cavity, a top member with side walls and end walls integrally connected to a top wall to define a cavity, a flange extending outward from said side walls and end walls, said flange defining a step therein which fits over said flange of said bottom member.

20. A container assembly as claimed in claim 19 wherein said inner tray lid is provided with a base and a pull tab mounted to said base.
